(19) United States
(12) Patent Application Publication
(10) Pub. No.: US 2005/0074483 A1
(43) Pub. Date: Apr. 7, 2005

(54) Pre-packaged multi-step skin care system

(75) Inventor: Beth A. Lange, Germantown, TN (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/679,566

(22) Filed: Oct. 6, 2003

(51) Int. Cl. A61K 7/00

(52) U.S. Cl. 424/443

(57) ABSTRACT

Pre-packaged multi-step skin care systems are disclosed wherein numerous products are bundled together that provide skin health benefits along the skin care continuum. The products, which can be wet wipes, are marked in such a manner as to instruct the user as to the preferred sequence of use of the products to attain maximum benefit therefrom. The products may be marked with a numeric system, an alpha numeric system, a word system, and/or a coloring system to instruct the user.

### BACKGROUND OF THE INVENTION

The present invention relates to a skin care system. More particularly, the present invention relates to a pre-packaged multi-step skin care system. The system comprises a plurality of skin care products for imparting multiple benefits to the skin. The products are marked in such a way so as to alert users to the proper sequence of use of the skin care products. In one embodiment, the skin care system provides a plurality of wet wipes that can effectively clean, maintain, and protect skin upon application and that are sequentially numbered to alert the user to the proper sequence of use.

In today's marketplace, a variety of personal care products are commercially available that are specifically designed for direct application to the skin. These products can typically be categorized in terms of the action they take upon the skin, and, to some extent, the layer of the skin they treat. For example, most products fall somewhere along the skin health continuum, which can be defined as (1) cleaning the skin; (2) maintaining the skin; (3) protecting and preventing damage to the skin; (4) treating the skin; and (5) diagnosing and monitoring the health of the skin.

Consumers have a myriad of choices when it comes to skin care products for direct application to the skin. Numerous types of products are commercially available for application and treatment of the skin. These products include, for example, wet wipes, dry wipes, foams, soaps, lotions, salves, gels, powders, cakes, and the like. Various products perform different functions when applied to the skin including, for example, cleaning the skin, treating the skin, protecting the skin, preventing damage to the skin, maintaining the health of the skin, etc. With so many types of products available that perform so many different functions on the skin, consumers are often confused about which types of products to buy, and how to properly use the products that they do buy. For example, one problem of delivering any one of the steps of the skin health continuum is that the consumer needs to understand which steps to take, and in what order to take them. Because many consumers may not know how important it is to clean the skin before treating it, they often purchase and apply the incorrect product to the skin. Even if the correct products are purchased, they may be applied by the user in the wrong sequence. By using products designed for different purposes in the wrong sequence, any benefits of any of the products on the skin can be lost or minimized. This can result in a disappointed consumer when the desired results on the skin are not achieved. Additionally, this can result in a reduced amount of consumer re-purchases of products which reduces overall product sales.

Based on the foregoing, there exists a need in the skin care industry for an easy to use, prepackaged skin care system which provides a plurality of skin care products that can provide numerous skin care benefits along the skin care continuum to a consumer in a preferred order to allow maximum benefit to the skin. Additionally, it would be advantageous if the skin care system provided easy to use instructions to alert the consumer to the proper order of usage of the products of the system.

### SUMMARY OF THE INVENTION

The present invention relates to an easy to use, pre-packaged multi-step skin care system which can provide numerous skin care benefits along the skin care continuum to a user in the proper sequential order and minimize or eliminate user error and confusion. In one embodiment, the skin care system comprises a plurality of wet wipes, which are designed to provide multiple benefits, sequentially numbered to alert the user to a preferred order of use of the wipes. Each of the wet wipes of the system provides a different type of benefit to the skin. By using the wet wipes in the proper order, the skin health of the user is improved as multiple benefits along the skin health continuum are realized.

In another embodiment, the multi-step skin care system comprises a plurality of wet wipes which are color coded to alert the user to a preferred order of use of the wipes. Alternatively, the packaging incorporating the wet wipes, or other products described herein, can be sequentially numbered and/or color coded to alert the user to a preferred order of use of the products.

The pre-packaged multi-step skin care system described herein provides users with numerous benefits as compared to currently commercially available products. Some benefits include, for example: (1) improved physiological/health/functional outcomes due to a systems approach; (2) time savings by having numerous products bundled together in a single package; (3) improved user understanding of product usage, including dosing needs and the prevention of overdosing and underdosing; and (4) an improved system for the delivery of skin care products by a caregiver to the elderly and children.

Briefly, therefore, the present invention is directed to a pre-packaged multi-step skin care system for caring for skin. The system comprises a first wet wipe for imparting a first benefit to the skin, a second wet wipe for imparting a second benefit to the skin, and a third wet wipe for imparting a third benefit to the skin. The first wet wipe comprises a marking to alert a user to use the first wet wipe prior to the second wet wipe, the second wet wipe comprises a marking to alert a user to use the second wet wipe after the first wet wipe, and the third wet wipe comprises a marking to alert a user to use the third wet wipe after the second wet wipe.

The present invention is further directed to a pre-packaged multi-step skin care system for caring for skin. The system comprises a first container comprising a first wet wipe for imparting a first benefit to the skin, a second container comprising a second wet wipe for imparting a second benefit to the skin, and a third container comprising a third wet wipe for imparting a third benefit to the skin. The first wet wipe comprises a marking to alert a user to use the first wet wipe prior to the second wet wipe. The second wet wipe comprises a marking to alert a user to use the second wet wipe after the first wet wipe, and the third wet wipe comprises a marking to alert a user to use the third wet wipe after the second wet wipe.

The present invention is further directed to a pre-packaged multi-step skin care system for caring for skin. The system comprises a first container comprising a first wet wipe for imparting a first benefit to the skin, a second container comprising a second wet wipe for imparting a second benefit to the skin, and a third container comprising a third wet wipe for imparting a third benefit to the skin. The first container comprises a marking to alert a user to use the first wet wipe prior to the second wet wipe. The second container comprises a marking to alert a user to use the second wet wipe after the first wet wipe, and the third container comprises a marking to alert a user to use the third wet wipe after the second wet wipe.

The present invention is further directed to a pre-packaged multi-step skin care system for caring for skin. The system comprises a first product, a second product, and a third product. The first product comprises a marking to alert a user to use the first product prior to the second product, the second product comprises a marking to alert a user to use the second product after the first product, and the third product comprises a marking to alert a user to use the third product after the second product.

The present invention is further directed to a pre-packaged multi-step skin care system for caring for skin. The system comprises a first wet wipe for cleaning the skin, a second wet wipe for moisturizing the skin, and a third wet wipe for applying sunscreen to the skin. The first wet wipe comprises a marking to alert a user to use the first wet wipe prior to the second wet wipe, the second wet wipe comprises a marking to alert a user to use the second wet wipe after the first wet wipe, and the third wet wipe comprises a marking to alert a user to use the third wet wipe after the second wet wipe.

Other features and advantages of this invention will be in part apparent and in part pointed out hereinafter.

### DETAILED DESCRIPTION OF THE PREFERRED EMBODIMENTS

In accordance with the present invention, it has been discovered that consumer confusion regarding the use of skin care products can be minimized or eliminated by providing the consumer with a pre-packaged multi-step skin care system. The pre-packaged multi-step skin care system may comprise a plurality of products, such as wet wipes, each designed to impart a different benefit onto the skin, and each clearly marked in such a way as to direct the user to use the products in the proper sequential order.

The pre-packaged multi-step skin care systems described herein may comprise various types of skin care products which may deliver one or more benefits along the skin care continuum. The benefits may include cleaning the skin, maintaining the skin, protecting and preventing damage to the skin, treating the skin, and diagnosing and monitoring the health of the skin. As used herein, the term “pre-packaged” means a single bundled package for sale to a user which may comprise only one individual container, or may comprise more than one container, such as two, three, four, or more containers. For example, in one embodiment of the present invention, a pre-packaged system includes only a single container comprising a number of wet wipes. In another embodiment, a pre-packaged system includes three separate containers, with each container comprising a different type of wet wipe.

Typical types of skin care products which can be used in the pre-packaged, multi-step skin care systems of the present invention include, for example, wet wipes, dry wipes, gloves, finger cots, poufs, cotton balls, lotions, creams, salves, ointments, patches, Sebutape, D-squame tape, Sellotape, blotter paper, indicators such as pH indicators and temperature indicators, and combinations thereof. Particularly preferred skin care products for use in the skin care systems of the present invention include wet wipes.

The pre-packaged multi-step skin care systems described herein comprise various products bundled and sold together, in a single package, for use on the skin in a prescribed sequence. In one embodiment, the single package may comprise a single skin care system designed for a single use or application to the skin; that is, the single package for sale to the user may comprise three products designed for a single application to the skin. A specific example of this type of packaging includes a single package comprising three wet wipes; one for cleaning the skin, a second for maintaining the skin, and a third for protecting and preventing damage to the skin.

In an alternative embodiment, the pre-packaged multi-step skin care system for sale to the user may comprise multiple containers with each container being designed for supporting a single application to the skin. In this embodiment, the single package for sale to the user may comprise 20 individually packaged skin care systems. Each skin care system may comprise a first wipe for cleaning the skin, a second wipe for maintaining the skin, and a third wipe for protecting and preventing damage to the skin.

In an alternative embodiment, the pre-packaged multi-step skin care system may be designed to allow for multiple applications to the skin; that is, the single package for sale to the user may comprise three separate containers of wet wipes, with each separate container containing multiple wipes. Specifically, each container of wet wipes contained in the pre-packaged multi-step skin care system may include 68 wet wipes. As such, the single package for sale to the consumer would comprise three separate individual containers, each containing 68 wet wipes to allow for multiple applications of the system.

In another embodiment, the pre-packaged multi-step skin care system may comprise wet wipes for imparting the desired benefits onto the skin, and may comprise a single container for sale to the user. This single container may consist of multiples of each wipe set in a prescribed sequential order in the container. For example, if the skin care system comprised a first wet wipe for cleaning the skin, a second wet wipe for maintaining the skin, and a third wet wipe for protecting and preventing damage to the skin, the single package for sale to the user may comprise 21 wipes introduced into the package in the prescribed order or use. That is, the first wipe would be a wipe for cleaning the skin, the second wipe would be a wipe for maintaining the skin, the third wipe would be for protecting and preventing damage to the skin, the fourth wipe would be for cleaning the skin, etc.

Although discussed primarily herein with respect to wet wipes, it will be recognized by one skilled in the art based on the disclosure herein that any number of the above-noted products could be substituted for wet wipes in any number of the packaging arrangements described herein. For example, a single package for sale to the user could include a package of 68 wet wipes for cleaning the skin, a bottle of lotion for maintaining the skin, and a package of 68 wet wipes for protecting and preventing damage to the skin.

The multi-step skin care system described herein not only provides at least three products for application to the skin in a specified order, but also alerts the user to the proper sequence of use of the products to attain maximum skin benefit by individually marking each component to eliminate confusion about how to properly use the system. In one embodiment, each component of the skin care system sold together in a single package is marked with a number directly on the component (numeric system) to alert the user to the order in which the component should be utilized to gain maximum benefit to the skin. For example, if the skin care system included a first wet wipe for cleaning the skin, a second wet wipe for maintaining the skin, and a third wet wipe for protecting and preventing damage to the skin, each wipe would be marked with a number corresponding to the prescribed sequence of use. Specifically, the wet wipe for cleaning the skin would have the number “1” introduced onto it, the wet wipe for maintaining the skin would have the number “2” introduced onto it, and the wet wipe for protecting and preventing damage to the skin would have the number “3” introduced onto it. Alternatively, any individual package comprising one type of wet wipe (or other product type, such as a cream or lotion) could be marked with the appropriate number in place of, or in addition to, marking the actual wet wipe. That is, if the single package for sale to the consumer comprised three separate containers of different wipe types, each separate container could be marked with a number in place of, or in addition to, marking the wet wipe.

In another embodiment, letters (alpha-numeric system) could be introduced onto the wet wipes and/or packaging containing the wet wipes (or other products) of the skin care system to designate the prescribed sequence of use. For example, if the skin care system included a first wipe for cleaning the skin, a second wipe for maintaining the skin, and a third wipe for protecting the skin, the first wipe could have the letter “A” introduced onto it, the second wipe could have the letter “B” introduced onto it, and the third wipe could have the letter “C” introduced onto it to designate the prescribed sequence of use to the user. Additionally or alternatively, the packaging could have the letter introduced thereon.

In another embodiment, the product and/or container comprising the product could have introduced thereon entire words to designate the prescribed sequence of use of the products. For example, if the skin care system included a first wipe for cleaning the skin, a second wipe for maintaining the skin, and a third wipe for protecting the skin, the first wipe could have the word “cleaning” introduced onto it, the second wipe could have the word “maintaining” introduced onto it, and the third wipe could have the word “protecting” introduced onto it to designate the preferred sequence of use of the wipes. Additionally, the wipes or containers comprising the wipes could have a number introduced thereon in combination with an entire word. In this embodiment, the first wipe would have introduced thereon the number “1” and the word “cleaning,” etc. Instructions for use of the system would be introduced onto the packaging.

In another embodiment, the products and/or containers comprising the products of the pre-packaged multi-step skin care system of the present invention can be marked to designate the prescribed sequence of use through a coloring system. For example, if the skin care system included three separate containers of wipes for use in a designated sequence, each container containing the wipes could be colored differently to alert the user that each container contained a different type of wipe. In this embodiment, where the containers are colored differently to designate the prescribed sequence of use of the products, the packaging would typically contain instructions instructing the user on the proper sequence of use of the colors. For example, the first container of wet wipes may contain wipes for cleaning the skin and be colored red, the second container may contain wipes for maintaining the skin and may be colored white, and the third container may contain wipes for protecting and preventing damage to the skin and may be colored blue. Each of the three containers would also have instructions for the user describing the sequential use of the wipes as red first, then white, and finally blue to achieve maximum benefit. As will be understood by one skilled in the art, all of the packages and containers described herein for use in the skin systems of the present invention will typically include instructions on the use of the system.

In another embodiment, the wipes alone, or in combination with the packaging, can be color coded to alert the user as to the proper sequence of use. As noted above, the packaging would typically have instructions alerting the user to a preferred sequence of use of the wet wipes, or other products.

In another embodiment, the pre-packaged multi-step skin care system can comprise three separate containers and can alert the user to the proper sequence of use of the products by varying the size of the containers comprising the products. For example, if the system comprised a first container of wet wipes for cleaning the skin, a second container for maintaining the skin, and a third container for protecting and preventing damage to the skin, the sizes of the containers and/or the products could be varied to alert the user that each container contains a separate type of wipe. The first container containing the cleaning wipes may be of a large size, the second container containing the maintaining wipes may be of a medium size, and the third container containing the protecting and preventing damage wipes may be of a small size. Typically, each individual container would contain instructions of use for the user on which size contained which wipe.

Based on the disclosure herein, it will be apparent to one skilled in the art that any one or more of the steps comprising the skin care systems described herein can be accomplished through the use of more than one product in combination, as opposed to a single product for each step. Specifically, if the skin care system is designed to comprise three separate steps of cleaning the skin, maintaining the skin, and protecting the skin, the first step (and/or any other step or steps) may be accomplished through the use of a combination of two or more products. For example, the first step of cleaning may be accomplished using a first wet wipe comprising a surfactant for cleaning the skin followed by a separate dry wipe for drying the skin prior to the second step of maintaining the skin. If the second step of maintaining was carried out using a single wet wipe and the third step of protecting was carried out using a single wet wipe, the system would comprise four separate wipes; one wet wipe and one dry wipe for the cleaning step, another wet wipe for the maintaining step, and another wet wipe for the protecting step.

Based on the disclosure herein, it will be apparent to one skilled in the art that there are multiple combinations of products and marking methods that could be employed to produce pre-packaged multi-step skin care systems as described herein to alert the user as to the proper sequence of use of the multiple products to attain the maximum benefit of the products. Although it is not possible to include a listing of all possible combinations, some particularly preferred combinations are set forth herein as examples of suitable skin care systems.

One preferred embodiment of the skin care systems described herein includes a pre-packaged multi-step system comprising three separate containers of wet wipes. The first wet wipe container contains wipes for cleaning the skin. Each wipe in the first container has introduced onto it the number “1” to indicate that it should be used prior to the other wipes. The second wet wipe container contains wipes for maintaining the skin. Each wipe in the second container has introduced onto it the number “2” to indicate that it should be used after the first wet wipe and prior to the third wet wipe. The third wet wipe container contains wipes for protecting and preventing damage to the skin. Each wipe in the third container has introduced onto it the number “3” to indicate that it should be used after the second wipe. All of the containers containing the wet wipes include directions for using the skin care system in sequential order.

Another preferred embodiment of the skin care systems described herein includes a pre-packaged multi-step system comprising three separate containers of wet wipes. The first wet wipe container contains wipes for cleaning the skin. Each wipe in the first container has introduced onto it the number “1” to indicate that it should be used prior to the other wipes. Also, the container itself is marked with a number “1” to indicate the proper sequence of use. The second wet wipe container contains wipes for maintaining the skin. Each wipe in the second container has introduced onto it the number “2” to indicate that it should be used after the first wet wipe and prior to the third wet wipe. Also, the container itself is marked with a number “2” to indicate the proper sequence of use. The third wet wipe container contains wipes for protecting and preventing damage to the skin. Each wipe in the third container has introduced onto it the number “3” to indicate that it should be used after the second wipe. Also, the container itself is marked with a number “3” to indicate the proper sequence of use. All of the containers containing the wet wipes include directions for using the skin care system in sequential order.

Another preferred embodiment of the skin care systems described herein includes a pre-packaged multi-step system comprising three wet wipes. The first wet wipe is for cleaning the skin and includes a number “1” on it to indicate that it should be used prior to the other two wet wipes in the system. The second wet wipe is for maintaining the skin and includes a number “2” on it to indicate that it should be used after the first wet wipe and prior to the third. The third wet wipe is for protecting and preventing damage to the skin and includes a number “3” on it to indicate that it should be used after the second wet wipe. The package comprising the three wet wipes includes directions on the sequential use of the wipes.

The pre-packaged multi-step skin care systems of the present invention may be suitable for use on all areas of skin on the body. Skin care systems can be custom designed to treat different areas of skin on the body including, for example, facial and neck skin, body skin, and perineal skin. Specifically, skin care systems could be custom designed for facial application, feminine hygiene uses and foot care.

As noted above, the pre-packaged multi-step skin care systems described herein can include a wet wipe for imparting a benefit along the skin care continuum to the skin of a user. Wipes suitable for use in the skin care systems comprise a wipe substrate in combination with a liquid formulation formulated to impart a specific benefit such as cleaning the skin, maintaining the skin, protecting and preventing damage to the skin, treating the skin or diagnosing and monitoring the health of the skin. The wipe substrate, or basesheet is generally rectangular in shape and may have any suitable unfolded width and length. For example, the wipe substrate may have an unfolded length of from about 2.0 centimeters to about 100.0 centimeters, and desirably from about 10.0 centimeters to about 25.0 centimeters, and an unfolded width of from about 2.0 centimeters to about 80.0 centimeters and desirably from about 10.0 centimeters to about 25.0 centimeters. Typically, each individual wipe substrate is arranged in a folded configuration and stacked one on top of the other to provide a stack of skin care wipes. Such folded configurations are well known to those skilled in the art and include c-folded, z-folded, quarter-folded configurations and the like. The stack of folded wipes may be placed in the interior of a container, such as a plastic tub, to provide a package of wipes for eventual sale to the consumer. Alternatively, the wipes may include a continuous strip of material which has perforations between each wipe and which may be arranged in a stack or wound into a roll for dispensing.

Optionally, the wipe may assume a variety of shapes, including but not limited to, generally circular, oval, square, or irregularly shaped depending upon numerous factors. The size of the wipe may also vary depending upon the desired end use of the wipe.

The materials of the substrate or basesheet, whether single or multi-layered, may be varied to provide different physical properties. The different physical properties, which a layer may be configured to provide, may be controlled by selecting the appropriate materials having characteristics such as softness, resiliency, strength, flexibility, integrity, toughness, absorbency, liquid retention, thickness, tear resistance, surface texture, drapability, wettability, wicking ability and the like, and combinations thereof. The wipe substrate can be configured to provide all desired physical properties within one layer, or configured to provide only specific physical properties within individual layers of a multi-layered wipe. For example, the wipe substrate may include at least one layer of material that is configured to provide strength and resilience to the wipe, and at least one other layer which is configured to provide a soft, gentle wiping surface to the wipe. Desirably, the wipes provide a soft wiping surface for contact with the skin and application of the skin care formulation.

The one or more layers of the wipe product can be made from a variety of materials including meltblown materials, coform materials, air-laid materials, bonded-carded web materials, hydroentangled materials, spunbond materials and the like, and can comprise synthetic or natural fibers. Examples of natural fibers suitable for use in the present invention include cellulosic fibers such as wood pulp fibers, cotton fibers, flax fibers, jute fibers, silk fibers and the like. Examples of thermoplastic polymeric fibers suitable for use with the present invention include polyolefins such as polypropylene and polyethylene, polyamides, and polyesters such as polyethylene teraphthalate. Alternative synthetic fibers which may be suitable include staple nylon and rayon fibers. The layer or layers of the wipe can be woven or nonwoven materials. In addition, the materials can be formed into balls, such as cotton balls, or applied to delivery systems such as applicators for swabs.

If one or more layers of the basesheet is a combination of polymeric and natural fibers, such as polypropylene and cellulosic fibers, the relative percentages of the polymeric fibers and natural fibers in the layer can vary over a wide range depending on the desired characteristics of the wipe. For example, the layer may comprise from about 20 to about 95 weight percent, desirably from about 20 to about 60 weight percent, and more desirably from about 30 to about 40 weight percent of polymeric fibers based on the dry weight of the layer. Such a layer of polymeric and natural fibers may be manufactured by any method known to those skilled in the art.

Generally, it is desirable that a layer comprising both polymeric and natural fibers be formed by a coform process for a more uniform distribution of the polymeric and natural fibers within the layer. Such coform layers are manufactured generally as described in U.S. Pat. No. 4,100,324 to Anderson et al. which issued Jul. 11, 1978; U.S. Pat. No. 4,604,313 to McFarland et al. which issued Aug. 5, 1986; and U.S. Pat. No. 5,350,624 which issued Sep. 27, 1994; which is herein incorporated by reference to the extent they are consistent herewith.

Typically, such coform layers comprise a gas-formed matrix of thermoplastic polymeric meltblown microfibers, such as, for example, polypropylene microfibers, and cellulosic fibers, such as, for example, wood pulp fibers. A coform layer is formed by initially forming at least one primary air stream containing the synthetic or polymeric fibers and merging the primary stream with at least one secondary stream of natural or cellulosic fibers. The primary and secondary streams are merged under turbulent conditions to form an integrated stream containing a thorough, homogeneous distribution of the different fibers. The integrated air stream is directed onto a forming surface to air form the layer of material. A multiplicity of these coform layers can then be formed in succession to provide a web of multiple coform layers.

The different fibers in the different layers of the layered basesheet of the present invention, such as the polypropylene and polyethylene microfibers set forth above, typically may not be compatible with and may not bond to each other. However, the different fibers may entangle with each other resulting in suitable securement between the layers. For example, in a layered basesheet containing a coform layer of polyethylene and cellulosic fibers and a coform layer of polypropylene and cellulosic fibers, the polyethylene and polypropylene fibers may entangle with each other and the cellulosic fibers and may at least partially bond to the cellulosic fibers which results in securement between the layers. Such interlayer bonding and entanglement may be enhanced by a thermo-mechanical process wherein the layered basesheet is passed between a heated smooth anvil roll and a heated pattern roll. The pattern roll may have any raised pattern which provides the desired entanglement and interlayer bonding. Desirably, the pattern roll defines a raised pattern which defines a plurality of bond locations which define a bond area of between about 4 and about 30 percent of the total area of the roll for improved interlayer attachment.

The basesheet for the wipe may have a total basis weight of from about 25 to about 120 grams per square meter and desirably from about 40 to about 90 grams per square meter. The basis weight of the basesheet may vary depending upon one or more desired characteristics of the wipe. For example, a suitable basesheet for wiping a skin care formulation onto the skin may define a basis weight of from about 60 to about 80 grams per square meter and desirably about 75 grams per square meter. In a particular embodiment wherein the basesheet includes coform layers of polypropylene and cellulosic fibers and polyethylene and cellulosic fibers, the layered basesheet defines a basis weight of from about 60 to about 90 grams per square meter and desirably about 80 grams per square meter, for improved softness and adequate strength.

In a particular embodiment, it is desired that the wipe of the present invention define sufficient strength to withstand the forces exerted by the user when it is used to apply the formulation to the skin. For example, the basesheet for the wipe may define a tensile strength of at least about 1.23 Newtons per centimeter in the machine direction and at least about 0.70 Newtons per centimeter in the cross machine direction. Wipes having alternate ranges of tensile strength may also be effectively employed in transferring a skin care formulation to the skin. As used herein, the term “machine direction” refers to the direction in which the material is manufactured while the cross machine direction refers to a direction which is perpendicular to the machine direction.

In a particular embodiment, wherein the basesheet includes coform layers of polypropylene and cellulosic fibers and polyethylene and cellulosic fibers, the layered basesheet defines a tensile strength of from about 1.31 to about 3.50 Newtons per centimeter in the machine direction and from about 0.84 to about 1.40 Newtons per centimeter in the cross machine direction, and desirably from about 1.58 to about 1.93 Newtons per centimeter in the machine direction and from about 0.93 to about 1.11 Newtons per centimeter in the cross machine direction. In such a configuration, the coform layer, which includes polypropylene fibers, provides the majority of the strength to the basesheet while the coform layer, which includes the polyethylene fibers, provides a soft surface for contact with the skin of the user. Thus, the tensile strength of such a layered basesheet is higher than the tensile strength of a single layer containing polyethylene fibers and polypropylene fibers.

The skincare formulations described herein are typically applied to the basesheet in an amount sufficient to achieve the desired transfer rates discussed herein without oversaturating the basesheet which may lead to unwanted pooling of liquid in the wipe container and wasted liquid. Specifically, the skincare formulation is desirably applied to the basesheet in an amount of from about 100% (by weight based on the weight of the dry basesheet) to about 1000% (by weight based on the weight of the dry basesheet), desirably from about 100% (by weight based on the weight of the dry basesheet) to about 500% (by weight based on the weight of the dry basesheet), and even more desirably from about 100% (by weight based on the weight of the dry basesheet) to about 300% (by weight based on the weight of the dry basesheet). The exact amount of formulation applied to the basesheet may vary depending upon the desired application rate and the basesheet being utilized to deliver the skincare formulation. Also, the exact amount of skincare formulation applied to the basesheet may depend upon the packaging utilized to deliver the wipe to the ultimate consumer. For example, if the wipes are to be delivered in a tub comprising numerous wipes, excess skincare formulation may need to be applied to the wipes to ensure coverage from the top of the tub to the bottom.

As mentioned above, in one embodiment of the pre-packaged multi-step skin care system of the present invention, the wet wipes include a number or letter to designate the proper sequential use of the products to ensure maximum benefit to the skin. The letter or number may be introduced onto the wet wipe in any manner suitable which will not interfere with the performance of the wet wipe. In one embodiment, the letter or number is inked onto the wet wipe substrate. The ink may either be aqueous or non-aqueous and may be suspended, dispersed or dissolved in a solvent, such as water, for application to the wipe substrate. Suitable inks for use on the wipe substrate will exhibit minimal bleeding on contact with the liquid wet wipe formulation.

Suitable inks may comprise a pigment, a cross linking polymer, a cross linking agent and, optionally, a solvent. It is generally preferred that the inks have a viscosity higher than that of water. The ink may be printed onto the substrate using any method known to those skilled in the art. Preferably, the ink is printed onto a substantially dry substrate and then allowed to dry before any liquid solution is applied to the substrate. The process of drying the ink onto the substrate can be accelerated by using heating, ultraviolet light, and microwave or infrared ovens.

In one embodiment, the ink is applied to the substrate using a flexographic printer. Flexographic printing is well known in the art. Specific details of this type of printing process can be found in the Reference Encyclopedia of Flexographic Equipment and Supplies by Robert P. Long, and in the Manual For Flexographic Inks by Cliff Woof.

Alternatively, the numbers or letters indicating the proper sequence of use for the wet wipes can be introduced by embossing the number or letter onto the wet wipe substrate. Suitable embossing procedures and processes are known to those skilled in the art.

As described above, in some embodiments, it may be desirable to color code the wet wipe substrate to designate the proper usage sequence. Wet wipe substrates can be easily colored using any cosmetically acceptable dye color, or through the use of a colored film introduced between layers of non-woven materials comprising the wipe substrate.

As noted herein, the first step of the skin care continuum that the skin care systems of the present invention address is cleaning the skin. As used herein, cleaning the skin is meant to include, for example, removing bacteria, yeast, dander, allergens, soil, contaminants, and the like from the stratum corneum, the outer layer of the skin's surface. Numerous commercially available wet wipes perform a cleaning function. A suitable formulation for use in combination with a wipe substrate for cleaning the skin is set forth in U.S. Pat. No. 6,204,208 (Krzysik et al.) and comprises a surfactant in combination with aloe vera. The surfactant may be selected from the group consisting of ethoxylated hydrogenated fatty oils, monosaccharides, monosaccharide derivatives, polysaccharides, polysaccharide derivatives, and combinations thereof. Additionally, suitable formulations for use on a wipe substrate for cleaning the skin are set forth in U.S. Pat. No. 6,432,429 (Maddern et al.), U.S. Pat. No. 5,910,455 (Maddern et al.), and U.S. Pat. No. 6,017,832 (Yahiaoui et al.).

As noted above, the second step of the skin care continuum that the skin care systems of the present invention address is maintaining the skin. As used herein, maintaining the skin is meant to include maintaining or improving the condition of the epidermis layer of skin. Many types of chemical agents and natural extracts can be used alone or in combination with a wipe substrate to maintain the skin. Examples include skin strength improving agents such as ascorbate, phytoestrogen, and insulin minetics, and antioxidants and metal chelators utilized to prevent oxidation of the skin or repair free radical damage. Oil control agents, such as clays or talc, could be utilized to maintain low surface sebum levels. Wet wipe products suitable for use to maintain the skin are set forth in U.S. Pat. No. 6,440,437 (Krzysik et al.) and may comprise an oil-in-water emulsion comprising a natural fat or oil, sterol or sterol derivative, humectant, emulsifying surfactant, and water.

The third step of the skin care continuum is protecting and preventing damage to the skin. As used herein, protecting and preventing damage to the skin includes protecting and preventing damage to the epidermis and dermis layers of the skin. Agents such as antimicrobials, sunscreens, skin protectants and rash relievers are suitable agents for protecting and preventing damage to the skin. In one embodiment, a wet wipe comprising a sunscreen agent can be included in the skin care systems of the present invention such that upon use of the wipe, the sunscreen is transferred to the skin of the user to protect and prevent damage to the skin.

The fourth step of the skin care continuum is treating the skin. As used herein, treating the skin is meant to include treating the skin with agents that improve the health of the skin and counteract the aging process. Typically, this means treating the dermis. The skin can be treated with various products, such as wet wipes and lotions, to counteract the effects of aging which can result in wrinkles, lines, sagging, hyperpigmentation, and age spots. For example, a wet wipe may be impregnated with a retinoid, a dermabrasion material, hydroquinone or other skin-lightening agents, and/or alpha-hydroxy acids to treat the skin. Additionally, treating the skin may include acne products such as benzyl peroxide, anti-inflammatories such as hydrocortisone, and topical anesthetics such as lidocaine.

The fifth step of the skin care continuum is diagnosing and monitoring the health of the skin. Diagnosing and monitoring the health of the skin is meant to include identifying problems and monitoring the condition of the skin. Suitable products for diagnosing and monitoring the health of the skin include wipes and lotions which comprise a biosensor, for example. In one example, a wet wipe can be used to introduce a composition onto the skin that can signal overexposure to the sun.

In view of the above, it will be seen that the several objects of the invention are achieved. As various changes could be made in the above-described skin care systems without departing from the scope of the invention, it is intended that all matter contained in the above description be interpreted as illustrative and not in a limiting sense.

1. A pre-packaged multi-step skin care system for caring for skin, the system comprising a first wet wipe for imparting a first benefit to the skin, a second wet wipe for imparting a second benefit to the skin, and a third wet wipe for imparting a third benefit to the skin, the first wet wipe comprising a marking to alert a user to use the first wet wipe prior to the second wet wipe, the second wet wipe comprising a marking to alert a user to use the second wet wipe after the first wet wipe, and the third wet wipe comprising a marking to alert a user to use the third wet wipe after the second wet wipe.

2. The skin care system as set forth in claim 1 wherein the marking on the first wet wipe, the second wet wipe, and the third wet wipe comprises a sequential numeric system.

3. The skin care system as set forth in claim 1 wherein the marking on the first wet wipe, the second wet wipe, and the third wet wipe comprises a sequential alpha-numeric system.

4. The skin care system as set forth in claim 1 wherein the marking on the first wet wipe, the second wet wipe, and the third wet wipe comprises a coloring system.

5. The skin care system as set forth in claim 1 further comprising a fourth wet wipe, the fourth wet wipe comprising a marking to alert a user to use the fourth wet wipe after the third wet wipe.

6. The skin care system as set forth in claim 1 further comprising a fifth wet wipe, the fifth wet wipe comprising a marking to alert a user to use the fifth wet wipe after the fourth wet wipe.

7. The skin care system as set forth in claim 1 wherein the first benefit imparted by the first wipe is a cleaning of the skin benefit.

8. The skin care system as set forth in claim 7 wherein the second benefit imparted by the second wipe is a maintaining of the skin benefit.

9. The skin care system as set forth in claim 8 wherein the third benefit imparted by the third wipe is a protecting and preventing damage benefit.

10. The skin care system as set forth in claim 1 wherein the pre-packaged multi-step skin care system comprises a single container comprising the first wet wipe, the second wet wipe, and the third wet wipe.

11. The skin care system as set forth in claim 1 wherein the pre-packaged multi-step skin care system comprises a first container comprising the first wet wipe, a second container comprising the second wet wipe, and a third container comprising the third wet wipe.

12. A pre-packaged multi-step skin care system for caring for skin, the system comprising a first container comprising a first wet wipe for imparting a first benefit to the skin, a second container comprising a second wet wipe for imparting a second benefit to the skin, and a third container comprising a third wet wipe for imparting a third benefit to the skin, the first wet wipe comprising a marking to alert a user to use the first wet wipe prior to the second wet wipe, the second wet wipe comprising a marking to alert a user to use the second wet wipe after the first wet wipe, and the third wet wipe comprising a marking to alert a user to use the third wet wipe after the second wet wipe.

13. The skin care system as set forth in claim 12 wherein the marking on the first wet wipe, the second wet wipe, and the third wet wipe comprises a sequential numeric system.

14. The skin care system as set forth in claim 12 wherein the marking on the first wet wipe, the second wet wipe, and the third wet wipe comprises a sequential alpha-numeric system.

15. The skin care system as set forth in claim 12 wherein the marking on the first wet wipe, the second wet wipe, and the third wet wipe comprises a coloring system.

16. The skin care system as set forth in claim 12 further comprising a fourth wet wipe, the fourth wet wipe comprising a marking to alert a user to use the fourth wet wipe after the third wet wipe.

17. The skin care system as set forth in claim 12 further comprising a fifth wet wipe, the fifth wet wipe comprising a marking to alert a user to use the fifth wet wipe after the fourth wet wipe.

18. The skin care system as set forth in claim 12 wherein the first benefit imparted by the first wipe is a cleaning of the skin benefit.

19. The skin care system as set forth in claim 18 wherein the second benefit imparted by the second wipe is a maintaining of the skin benefit.

20. The skin care system as set forth in claim 19 wherein the third benefit imparted by the third wipe is a protecting and preventing damage benefit.

21. A pre-packaged multi-step skin care system for caring for skin, the system comprising a first container comprising a first wet wipe for imparting a first benefit to the skin, a second container comprising a second wet wipe for imparting a second benefit to the skin, and a third container comprising a third wet wipe for imparting a third benefit to the skin, the first container comprising a marking to alert a user to use the first wet wipe prior to the second wet wipe, the second container comprising a marking to alert a user to use the second wet wipe after the first wet wipe, and the third container comprising a marking to alert a user to use the third wet wipe after the second wet wipe.

22. The skin care system as set forth in claim 21 wherein the marking on the first container, the second container, and the third container comprises a sequential numeric system.

23. The skin care system as set forth in claim 21 wherein the marking on the first container, the second container, and the third container comprises a sequential alpha-numeric system.

24. The skin care system as set forth in claim 21 wherein the marking on the first container, the second container, and the third container comprises a coloring system.

25. The skin care system as set forth in claim 21 wherein the first wet wipe, second wet wipe, and third wet wipe comprise a marking for alerting a user to the proper sequence of use of the wipes, the marking system comprising a sequential numeric system.

26. The skin care system as set forth in claim 21 wherein the first wet wipe, second wet wipe, and third wet wipe comprise a marking for alerting a user to the proper sequence of use of the wipes, the marking system comprising a sequential alpha-numeric system.

27. The skin care system as set forth in claim 21 wherein the first wet wipe, second wet wipe, and third wet wipe comprise a marking for alerting a user to the proper sequence of use of the wipes, the marking system comprising a coloring system.

28. A pre-packaged multi-step skin care system for caring for skin, the system comprising a first product, a second product, and a third product, the first product comprising a marking to alert a user to use the first product prior to the second product, the second product comprising a marking to alert a user to use the second product after the first product, and the third product comprising a marking to alert a user to use the third product after the second product.

29. The skin care system as set forth in claim 28 wherein the marking on the first product, the second product, and the third product comprises a sequential numeric system.

30. The skin care system as set forth in claim 28 wherein the marking on the first product, the second product, and the third product comprises a sequential alpha-numeric system.

31. The skin care system as set forth in claim 28 wherein the marking on the first product, the second product, and the third product comprises a coloring system.

32. The skin care system as set forth in claim 28 wherein the first product, second product, and third product are selected from the group consisting of gloves, finger cots, poufs, cotton balls, lotions, soaps, ointments, salves, patches, and combinations thereof.

33. A pre-packaged multi-step skin care system for caring for skin, the system comprising a first wet wipe for cleaning the skin, a second wet wipe for moisturizing the skin, and a third wet wipe for applying sunscreen to the skin, the first wet wipe comprising a marking to alert a user to use the first wet wipe prior to the second wet wipe, the second wet wipe comprising a marking to alert a user to use the second wet wipe after the first wet wipe, and the third wet wipe comprising a marking to alert a user to use the third wet wipe after the second wet wipe.

34. The skin care system as set forth in claim 33 wherein the marking on the first wet wipe, the second wet wipe, and the third wet wipe comprises a sequential numeric system.

35. The skin care system as set forth in claim 33 wherein the marking on the first wet wipe, the second wet wipe, and the third wet wipe comprises a sequential alpha-numeric system.

36. The skin care system as set forth in claim 33 wherein the marking on the first wet wipe, the second wet wipe, and the third wet wipe comprises a coloring system.

37. The skin care system as set forth in claim 33 further comprising a fourth wet wipe, the fourth wet wipe comprising a marking to alert a user to use the fourth wet wipe after the third wet wipe.

38. The skin care system as set forth in claim 33 further comprising a fifth wet wipe, the fifth wet wipe comprising a marking to alert a user to use the fifth wet wipe after the fourth wet wipe.

39. The skin care system as set forth in claim 33 wherein the pre-packaged multi-step skin care system comprises a single container comprising the first wet wipe, the second wet wipe, and the third wet wipe.

40. The skin care system as set forth in claim 33 wherein the pre-packaged multi-step skin care system comprises a first container comprising the first wet wipe, a second container comprising the second wet wipe, and a third container comprising the third wet wipe.
